(19) United States
(12) Patent Application Publication
(10) Pub. No.: US 2009/0191281 A1
(43) Pub. Date: Jul. 30, 2009

(54) PHARMACEUTICAL COMPOSITION, PROCESS FOR MANUFACTURING THE SAME AND ITS MEDICAL DEVICE FOR THE TREATMENT OF CUTANEOUS LESIONS

(75) Inventor: Alfred MARCHAL, Waterloo (BE)

Correspondence Address: SUGHRUE MION, PLLC, 2100 PENNSYLVANIA AVENUE, N.W., SUITE 800, WASHINGTON, DC, 20037, US

(73) Assignee: AURIGA INTERNATIONAL, Waterloo (BE)

(21) Appl. No.: 12/358,803

(22) Filed: Jan. 23, 2009

(30) Foreign Application Priority Data
Jan. 25, 2008 (EP) 08100943.36

(51) Int. Cl. A61K 33/34; A61K 33/00; A61K 33/30

(52) U.S. Cl. 424/630; 424/718; 424/641

(57) ABSTRACT

Use, method of manufacturing a pharmaceutical composition, pharmaceutical composition for the treatment of skin ailments comprising a therapeutic quantity in aqueous solution of nitric acid of 5 to 15 mmol/ml of pharmaceutical composition, and also comprising a zinc salt at a concentration of 0.01 to 0.015 mg of Zn²⁺ ions/ml as well as a copper salt at a concentration of 0.01 to 0.015 mg of Cu²⁺ ions/ml of pharmaceutical composition and its medical device.

The present invention relates to a pharmaceutical composition for the treatment of skin ailments, comprising a therapeutic quantity in aqueous solution of nitric acid of 5 to 15 mmol/ml of pharmaceutical composition. Such a composition is for example known from the document “The revival of nitric acid for the treatment of anogenital warts, C L Heaton et al., Clinical Pharmacology & Therapeutics—July 1993-107, 111”, which discloses the advantages of the use of nitric acid in such lesions.

An aqueous solution of 6 to 10 M nitric acid that contains a metal nitride or a nitrous acid is known from the document EP 026 532. The solution according to EP 026 532 makes it possible to obtain a chemical reaction as effective as that of concentrated nitric acid, but without deploying the powerful caustic effect, in particular on tissues, of the latter.

Products as disclosed in EP 026 532 are manufactured by the addition of oxidisable organic acid to nitric acid. The oxidation of organic acids promotes the reduction of the nitric acid by promoting the formation of nitrate reduction products such as nitrous gases and nitrous acid and condensation products such as O-nitryl and O-nitrosyl derivatives.

Unfortunately, the solution produced according to the teaching of the document EP 026 532 is not stable over time and in addition its efficacy decreases when the concentration of nitrous derivative decreases.

According to the document EP 630 650, which describes an improvement to the EP 026 532 product by the same applicant, it is learnt that the efficacy of the product according to EP 026 532 is lost when the proportion of reaction products measurable as nitrite decreases too greatly. It is also learnt that the products obtained according to EP 026 532 require not hermetically closing the packages containing the products, but keeping them in a receptacle with loose closure.

Consequently, EP 026 532 uses various organic carboxylic acids that oxidise at different rates in order to compensate for a reduction in the nitrite content.

According to EP 630 650, the reaction of the oxydisable carboxylic acids takes place too slowly, at ordinary temperature, to suffice to replace the nitrites and, to mitigate the fact that the nitrite concentration may undergo considerable variations according to temperature and duration of storage, provision is made for preparing a 1 to 5.5 M aqueous nitric acid solution and a primary alkanol.

According to EP 630 650, a solution is then obtained having an efficacy that is as good and fully reproducible, without incurring the disadvantages of the products already known, by virtue of this solution, which also makes it possible to slightly reduce the nitric acid concentration. Since stability is improved, there is a reduction in the risks due to the products that have become ineffective and have an increased danger of secondary effects and may in particular lead to ulcerations of healthy skin.

EP 026 532 also teaches the use of various metal salts such as those of copper, silver, cadmium, zinc, aluminium, calcium, strontium, magnesium, iron, antimony, bismuth, selenium, manganese, zirconium, cobalt, gold, titanium and zinc and prefers the corresponding nitrates.

Among these metal ions, copper, silver, cadmium and zinc ions are preferred without any distinction between them and without associating any advantageous technical effect with their presence.

One aim of the invention is to procure a novel pharmaceutical composition having an improved efficacy compared with the existing ones in the treatment of skin ailments and cutaneous lesions and which is also more stable over time, without however adding several active substances which might be considered to be problematical in modern pharmacology.

For this purpose, there is provided according to the invention a pharmaceutical composition as mentioned at the start also comprising a zinc salt at a concentration of 0.01 to 0.015 mg of zinc ions/ml of pharmaceutical composition as well as a copper salt in a quantity ranging 0.01 to 0.015 mg copper ions/ml of pharmaceutical composition.

Apart from its advantageous antiseptic and antibacterial effect, the copper conjointly with the nitric acid and the nitrites denatures the proteins of the skin and allows mummification of the lesions or skin ailments (necrosis of the area comprising the lesions).

The zinc has an action on cell immunity and therefore promotes the combating of infections, in particular in the cells of the human body and therefore also those of the skin.

Adding zinc to a composition for treating cutaneous lesions and skin ailments is particularly advantageous through its efficacy in increasing the cell immune response. In addition, zinc also has key role in the cell membranes and fulfills an important structural role, reinforcing the mediating effect of the cell immune response.

Consequently the pharmaceutical composition according to the invention promotes the cell response vis-á-vis a potential infection which might accompany the cutaneous lesion and reinforces the membrane structure of the skin cells. In addition, zinc promotes healing of the skin that suffered the said lesion, which makes its role completely synergetic with that of the nitric acid of the pharmaceutical composition according to the invention.

In such skin ailments or cutaneous lesions, the cells of the skin proliferate in an uncontrolled fashion in some cases while in others it is dead cells that accumulate on the surface of the skin.

It was therefore found surprisingly that, conjointly with the nitric acid that destroys the “abnormal” cells of the lesion or ailing cells, zinc had a beneficial effect on the lesion.

The result is particularly surprising since zinc is essential to cell growth and to multiplication of cells. Consequently it was rather unimaginable to use it when it is necessary to cease uncontrolled proliferation or when it is necessary to destroy accumulated cells in a tumour or in keratoses since it rather serves conventionally for their development.

It was shown by tests that zinc, unlike what might have been thought, had no effect contrary to that of nitric acid and that in addition healing after destruction of “abnormal” cells was more rapid and more effective and that the new skin cells, generally recognisable, had a reduced “new and red” appearance.

More particularly, as mentioned above, the concentration of zinc and copper ions in the composition according to the invention is similar in order to avoid competitions between the zinc and copper (side by side in Mendeleev's periodic table of elements) within the healing process. Copper and zinc are two elements catalysing the degradation of nitric acid within tissues, but copper contributes to obtaining necrosis by denaturation of proteins.

As mentioned above, copper, conjointly with nitric acid and nitrites, denatures the skin proteins and allows mummification of the skin lesions or ailments (necrosis of the area containing the lesion). It is therefore the presence of Zn, Cu and NO₂ (coming from the reduction of HNO₃) that acts during application in the skin by catalysing the destruction of the cutaneous lesion (for example the wart) by HNO₃. This is added to the other properties of Zn and Cu already mentioned.

Zinc, having chemical properties close to those of copper (similar atomic weights and electropositive potentials, etc), has a physiological role very close to that of copper, in particular vis-á-vis interactions with proteins. It is therefore advantageous for these two elements in ionised form to have a relative concentration in the composition according to the invention close to each other since they provide equilibrium. If the zinc concentration were greater than that of copper, the zinc could take the role of copper in the necrosis of the tissues and the copper would remain on the surface, which would undeniably cause hyperpigmentation of the cells of the post-cure epidermis. This is because copper, although having an antiseptic and antibacterial action, is also a catalyst for the manufacture of melanin, which causes the appearance of brown spots.

In addition, the composition according to the invention is obtained from metallic zinc (and therefore having a zero oxidation state), particularly appropriate for its effect as powerful reducer, which makes it possible to keep the nitrate concentration stable over time in the composition according to the invention. Consequently the composition according to the invention has made it possible to achieve great stability of the composition and an optimum healing and antiseptic effect while having an optimum necrosis effect and very much reduced secondary effects.

Advantageously, the pharmaceutical composition also comprises lactic acid in a quantity ranging from 1 to 50 mg/ml of pharmaceutical composition.

The pharmaceutical composition according to the invention comprises nitrates that are involved in the cascade of the reduction products of the nitrate that results from the interaction of concentrated nitric acid as oxidant with organic acids as reducers.

With the nitric acid, the organic acids therefore form reduction products that contribute to the presence of the minimal nitrate concentration in the composition and therefore make it possible, even in cases of instability, to produce the therapeutic concentration. This is because the nitrites constituting the products of the reduction of the nitrates are involved in two destruction mechanisms, the digestion or erosion of the tissue by acid hydrolysis of the peptide bonds and the devitalisation of the tissues by a covalent reaction with the proteins of the tissue, leaving the architecture of the lesion broadly preserved.

In addition, the pharmaceutical composition comprises, in one advantageous embodiment, oxalic acid in a quantity ranging from 10 to 70 mg/ml of pharmaceutical composition and/or acetic acid in a quantity ranging from 1 to 50 mg/ml of pharmaceutical composition.

Such a mixture of organic acids adds to the composition of the compounds that are oxydisable at different speeds. The acetic acid is relatively oxidised whereas the oxalic acid is oxidised very slowly. This mixture makes it possible to maintain a stable average of nitrous compounds, conjointly with the reducing zinc, which has a role in the rapid devitalisation of the lesions treated topically (C L Heaton et al), since the nitrite concentration is correlated with the yellowing of the tissues, which is an indicator of efficacy.

In addition, the presence of zinc in the pharmaceutical composition reduces the precipitation of the organic acids when they are present, which makes the presence of zinc even more advantageous.

In a preferential embodiment, the copper salt is a copper nitrate and preferably a copper(II) nitrate trihydrate.

In a variant according to the invention, the composition according to the invention also comprises zinc nitrate in order to further increase the nitrate content of the composition and consequently its efficacy in treating cutaneous lesions.

Other embodiments of the pharmaceutical composition according to the invention are mentioned in the accompanying claims.

The invention also relates to a method of manufacturing a pharmaceutical composition for the treatment of skin ailments comprising a therapeutic quantity in aqueous solution of nitric acid of 5 to 15 mmol/ml of pharmaceutical composition, comprising:
addition of a quantity of metallic zinc at a concentration of 0.01 to 0.015 mg of Zn²+ ions/ml to a predetermined quantity of concentrated nitric acid,
stirring of the said predetermined quantity of nitric acid containing zinc until the lafter is dissolved,
release of gaseous hydrogen with a reduction of nitric acid into nitrous acid and the formation of a first solution,
addition of a quantity of copper, in particular in the form of a copper nitrate, at a concentration of 0.01 to 0.015 mg of Cu²+ ions/ml, and
dilution with water.

As already mentioned above, through the fact that the composition according to the invention is obtained from metallic zinc (and therefore having a zero oxidation state) particularly appropriate for its powerful reducing effect, the nitrite concentration is kept stable in the course of the storage time of the composition according to the invention without adding pharmaceutical product or active substances requiring lengthy, expensive and laborious validation procedures.

The method according to the invention provides solely for the addition of zinc, which also makes it possible to achieve synergy with the copper in the denaturation of the proteins and the necrosis of unhealthy tissues as well as improving the healing effects.

In an advantageous form, the said addition of copper is carried out in the said first solution. In a preferential variant, the said addition of copper is carried out in another predetermined quantity of concentrated nitric acid with the formation of a second solution and is followed by a mixing of the first solution in the second solution before dilution with water.

Preferably, the method according to the invention also comprises an addition of at least one other carboxylic acid, preferably chosen from acetic acid, oxalic acid and lactic acid, and reduction of the said at least one carboxylic acid with the formation of nitrites.

Consequently the reducing conditions that contribute to the stable nitrite concentration are maintained over time by the differential and progressive oxidation of one or more of these carboxylic acids added according to the invention.

Other embodiments of the method according to the invention are mentioned in the accompanying claims.

Another object of the invention is a medical device comprising at least one applicator and an ampoule containing the pharmaceutical composition according to the invention.

The nitric acid contained in the pharmaceutical composition has a destructive effect on the cells, which is advantageous on the unhealthy cells but which would be prejudicial if it were applied to the healthy cells surrounding the lesion or the unhealthy cells. Likewise, the presence of copper ions could cause an undesirable hyperpigmentation on the healthy cells if the application of the product is not targeted.

It is therefore particularly advantageous to market an assembly comprising the said applicator and an ampoule, preferably made from glass and sealed, which contains the said composition in order to allow precise application of the composition. In addition, it is not appropriate to expose the composition according to the invention to oxidising open air.

Nitric acid being a corrosive acid, containing it in glass is advantageous. In addition, such an ampoule is easily sealed by flame, thus isolating it from the oxidising atmosphere that might have a detrimental effect on the aforementioned reduction product cascade of the nitrate.

In the present case, the composition according to the invention is completely isolated from the surrounding environment and the ampoule of the device according to the invention does not allow the passage of gas produced, thus maintaining the reducing conditions.

In an advantageous embodiment, the said applicator is a glass pipette arranged so as to take off the said pharmaceutical composition from the ampoule with a view to its application to a cutaneous lesion. This application is useful in the case of large lesions or when the ampoule is a receptacle containing a quantity of pharmaceutical composition suitable for several applications, and the glass pipette can easily be autoclaved and can be used on several occasions.

In a variant, the said applicator comprises a reservoir consisting of the said ampoule and an application end formed by a capillary having an end chosen from a bevelled end, a truncated part, a brush, a painting piece, an applicator pad, a hollow needle and the like. According to the required application (small lesion or larger lesion), the practitioner can either choose from several application end pieces or prescribe the appropriate end piece for his patient having a particular lesion.

In an alternative embodiment, the said applicator comprises a reservoir arranged to receive the content of the said ampoule and an application end formed by a capillary having an end chosen from a bevelled end, a truncated part, a brush, a painting piece, a hollow needle and the like.

In this embodiment, use of the medical device is particularly simplified and safe. This is because the ampoule is fixed to the said applicator like a cartridge on a ballpoint, which makes the assembly secure. The risks of tipping, overflow and leakages are reduced, giving rise to reduced risks of burning for the hands of the doctor.

For example, in the case of an applicator end piece, provision may be made for a simple pressure on the applicator pad to bring the appropriate quantity of composition according to the invention into the capillary and to allow the application of the solution to the area to be treated. The pad at the end of the capillary then prevents the solution from overflowing out of the area to be treated and prevents any risk of injuring the treated patient, in particular if the latter moves during the application of the solution. For example, in the case of children, it is frequent that the latter are frightened and move abruptly, which may have the result that they are injured by the glass end piece of the capillary. If this is provided with an applicator pad soaked with the solution according to the invention, the risk of injury is greatly reduced.

Other embodiments of the device according to the invention are mentioned in the accompanying claims.

The invention also relates to a use of the pharmaceutical composition as a medication for the treatment of skin ailments or cutaneous lesions such as dermatoses, keratoses, warts, condylomas, eczema, hyperkeratoses, acne, psoriasis, lesions resulting from fungal, bacterial or viral infections and the like.

Other characteristics, details and advantages of the invention will emerge from the description given below of a non-limitative example embodiment.

### EXAMPLE 1

### Manufacture of the Composition According to the Invention

2.75 g of oxalic acid dihydrate (2.75% mN—Prolabo, Pa.) and 5 mg of copper(II) nitrate trihydrate (Merck, Pa.—0.0050% mV) are placed in a hermetically closed flask.

Then 35.98 g of acid, that is to say 25.7 ml of 65% nitric acid (density 1.4), 1.72 ml of glacial acetic acid (Fisher Scientific, PA) and lactic acid DL to the extent of 167 μl (ALFA AESAR, ACS, 85 to 90%) were added.

Then in another flask 1.36 mg of metallic Zn (very pure Merck) was dissolved in 20 ml of 65% nitric acid (density 1.4) and stirring was maintained until the zinc was completely dissolved.

The content of the first flask was added to that of the second and the volume was increased to 100 ml with water and the flask was closed hermetically and mixing was carried out for 15 minutes. The solution was then re-cooled to room temperature.

Self-breakable ampoules of 200 μl were then filled and sealed under flame.

The ampoules are to be used in the following fashion.

The end piece is to be broken and the ampoule can then be fixed to the applicator.

The cutaneous lesion will first be cleaned with a conventional disinfection and the pharmaceutical composition according to the invention will be applied directly to the lesion with the applicator.

The bevelled end makes it possible to apply small quantities while the truncated end or brush makes it possible to treat larger lesions. To treat surfaces from 2 to 3 cm², it is preferable to use the glass pipette.

The solution will then be made to penetrate by a light pressure of the applicator on the lesion, which makes it possible to make the composition according to the invention penetrate solely in the epidermis and the dermis and polymerise in basal cells of the skin, making further penetration of the composition impossible. Consequently the repeated application at the same point reaches, as sought, only the upper layers of the skin, without damaging the basal cells of the tissue.

### EXAMPLE 2

### Evaluation of the Efficacy and Tolerance of the Composition According to the Invention as an Anti-Wart Treatment

23 patients aged from 18 to 63 years with an average age of 35.9 years were selected for a monocentric study. The patients each had one or more warts. This study lasted for 35 days. The composition according to the invention (containing 6.6 mmol/ml of HNO₃; 42 mg/ml of acetic acid, 35.7 mg/ml of oxalic acid, i.e. 0.397 mmol/ml; 4.5 mg/ml of lactic acid; 0.0126 mg/ml Cu²⁺ ions and 0.0134 mg/ml Zn ions) was applied after cleaning with alcohol and scarification by the doctor on the wart or warts at the rate of 1 to 3 applications at an interval of one week. The number of applications of the composition according to the invention was left to the assessment of the doctor responsible for the present clinical study.

Evaluation of Efficacy

The efficacy of the composition according to the invention was evaluated according to a clinical assessment of the patients at the end of the study (after 35 days). The doctor responsible for the present study visually examined the regression of the wart or warts on a scale from 0 to 3 (0 representing the case where no effect of the composition according to the invention is observed, 1 where the wart partially disappeared and 2 when the disappearance of the wart was total).

The results are illustrated in table 1.

TABLE 1

| Number of | | | |
|---|---|---|---|
| patients | Total cure | Partial cure | No effect |
| 23 | 12 | 8 | 3 |

As can be seen, only 3 patients did not respond at all to the treatment after three applications. Total cure was observed in 52% of the cases (12 patients) and 8 patients (34%) responded to the treatment partially. According to the doctors responsible for the study, if the treatment by the composition according to the invention had included additional applications, the number of total cures would have increased further.

In the case of the patients who showed total cure, it was also measured after how many applications total cure was observed. The results are illustrated in table 2.

TABLE 2

| | | 2 applications | 3 applications |
|---|---|---|---|
| | | spaced apart by 1 | spaced apart by 1 |
| Number of patients | 1 application | week | week |
| 12 | 2 | 6 | 4 |

As can be seen, in 50% of the cases, 2 applications spaced apart by a week suffice to obtain total cure of the warts.

Likewise, the influence of the location of the warts with respect to the result of the cure and the number of applications necessary for this cure was studied. No influence of the location of the warts, nor on the efficacy of the solution according to the invention, nor on the number of applications necessary for the total cure of the warts.

Evaluation of Tolerance

Overall, the composition according to the invention was very well tolerated. Some patients felt either tingling, or burning, but the doctors responsible for the present study concluded that there was very good tolerance of the composition according to the invention by the patients.

Naturally the present invention is in no way limited to the embodiments described above and many modifications can be made thereto without departing from the scope of the accompanying claims.

For example, provision is also made according to the invention for the composition also to comprise silver in ionised form for its major antiseptic effect. In this case the silver contributes to the good progress of the healing while preserving the tissue during healing from any infection.

In a similar manner, the preferential oxidising organic carboxalic acids used in the composition according to the invention are acetic acid, lactic acid and oxalic acid, but it goes without saying that others, such as glycolic acid, pyruvic acid, glycoxylic acid or malic acid and the like can also be used.

1. Pharmaceutical composition for the treatment of skin ailments, comprising a therapeutic quantity in aqueous solution of nitric acid of 5 to 15 mmol/ml of pharmaceutical composition, characterised in that it also comprises a zinc salt at a concentration of 0.01 to 0.015 mg of Zn²⁺ ions/ml as well as a solution of copper at a concentration of 0.01 to 0.015 mg of Cu²⁺ ions/ml of pharmaceutical composition.

2. Pharmaceutical composition according to claim 1, also comprising lactic acid in a quantity ranging from 1 to 50 mg/ml of pharmaceutical composition.

3. Pharmaceutical composition according to claim 1, also comprising oxalic acid in a quantity ranging from 10 to 70 mg/ml of pharmaceutical composition.

4. Pharmaceutical composition according to claim 1, also comprising acetic acid in a quantity ranging from 1 to 50 mg/ml of pharmaceutical composition.

5. Pharmaceutical composition according to claim 1, in which the said copper salt is a copper nitrate and preferably a copper(II) nitrate trihydrate.

6. Pharmaceutical composition according to claim 1, also comprising zinc nitrate.

7. Method of manufacturing a pharmaceutical composition for the treatment of skin ailments comprising a therapeutic quantity in aqueous solution of nitric acid of 5 to 15 mmol/ml of pharmaceutical composition, comprising:
addition of a quantity of metallic zinc at a concentration of 0.01 to 0.015 mg of Zn²⁺ ions/ml to a predetermined quantity of concentrated nitric acid,
stirring of the said predetermined quantity of nitric acid containing zinc until the latter is dissolved,
release of gaseous hydrogen with a reduction of nitric acid into nitrous acid and the formation of a first solution,
addition of a quantity of copper, in particular in the form of a copper nitrate, at a concentration of 0.01 to 0.015 mg of Cu²⁺ ions/ml, and
dilution with water.

8. Method according to claim 7, in which the said addition of copper is carried out in the said first solution.

9. Method according to claim 7, in which the said addition of copper is carried out in another predetermined quantity of concentrated nitric acid with the formation of a second solution and is followed by a mixing of the first solution with the second solution before dilution with water.

10. Method according to claim 7, also comprising an addition of at least one other carboxylic acid, preferably chosen from acetic acid, oxalic acid and lactic acid, and a reduction of the said at least one carboxylic acid with the formation of nitrites.

11. Medical device comprising an applicator and an ampoule, the said ampoule containing a pharmaceutical composition according to claim 1.

12. Medical device according to claim 11, in which the said applicator is a glass pipette arranged to take off the said pharmaceutical composition from the ampoule with a view to its application to a cutaneous lesion.

13. Medical device according to claim 11, in which the said applicator comprises a reservoir consisting of the said ampoule and an application end formed by a capillary having an end chosen from a bevelled end, a truncated part, a brush, a painting end piece, an applicator pad, a hollow needle and the like.

14. Medical device according to claim 11, in which the said applicator comprises a reservoir arranged to receive the content of the said ampoule and an application end formed by a capillary having an end chosen from a bevelled end, a truncated part, a brush, a painting end piece, a hollow needle and the like.

15. Use of the pharmaceutical composition according to claim 1 as a medication for the treatment of skin ailments or cutaneous lesions such as dermatoses, keratoses, warts, condylomas, eczema, hyperkeratoses, acne, psoriasis, lesions resulting from fungal, bacterial or viral infections and the like.
